(19) United States
(12) Patent Application Publication
(10) Pub. No.: US 2012/0258972 A1
(43) Pub. Date: Oct. 11, 2012

(54) Composition and Methods for Treating Hair Loss

(76) Inventors: Asif Rafi, Los Angeles, CA (US); Roger M. Katz, Marina Del Rey, CA (US)

(21) Appl. No.: 13/440,304

(22) Filed: Apr. 5, 2012

Related U.S. Application Data
(60) Provisional application No. 61/472,567, filed on Apr. 6, 2011.

(51) Int. Cl. A61K 31/506; A61P 31/10; A61K 31/496; A61P 17/14

(52) U.S. Cl. 514/254.07; 514/275

(57) ABSTRACT

A method for treating hair loss caused by androgenic alopecia and/or male pattern baldness. The method, which not only slows hair loss but causes hair re-growth, includes approximately daily application to a subject's scalp of a novel composition comprising finasteride (Propecia® or Proscar®), dutasteride (Avodart®), and minoxidil (Rogaine®) as active ingredients in a hypoallergenic cream-based vehicle, preferably coupled with daily ingestion of 1 mg per day of finasteride (Propecia® or Proscar®), application to the scalp of 5% minoxidil (Rogaine®) foam approximately once per day, and use of a ketoconazole-containing shampoo (e.g., Nizoral®) approximately 2-3 times per week. The method described herein also resolves scalp dermatitis in atopic subjects suffering therefrom. A method for making the novel composition is also provided.

### CROSS-REFERENCE TO RELATED APPLICATIONS

This patent application claims the benefit of U.S. Provisional Patent Application Ser. No. 61/472,567 filed Apr. 6, 2011 for Method for Treating Hair Loss, which application is incorporated in its entirety herein by this reference.

### BACKGROUND OF THE INVENTION

1. Technical Field

This invention relates to novel methods and a novel composition for treatment of hair loss by inducing hair growth in individuals suffering from androgenic alopecia.

More specifically, this invention relates to a novel regimen for inducing hair growth through a treatment approach involving topical application of the composition described herein. Said treatment approach can be supplemented with additional steps, which may further induce hair growth and/or limit hair loss. Said treatment approach depends, in part, on inhibition of one or both of the isozymes of the enzyme 5-alpha reductase, which converts testosterone to dihydrotestosterone. Dihydrotestosterone is believed to be responsible for androgenic alopecia, or male pattern baldness, in those who are genetically predisposed for such a condition.

2. Background Art

Androgenic alopecia, also called male pattern baldness, is a common phenomena, and is most commonly seen in men whose hair follicles are sensitive to dihydrotestosterone, which is the product of the testosterone-reducing enzyme 5-alpha reductase. Male pattern baldness affects approximately 30% of men by the age of 30, 50% of men by age 50, and 57% of men by age 60. See, Melia J. M., Perret M. C., Manzotti M., Catalano H. N., Guyatt G., Efficacy and Safely of Finasteride Therapy for Androgenetic Alopecia, Archives of Dermatology (2010) 146(10): 1141-1150; Ellis J. A., Sinclair R., Harrap S. B., Androgenetic Alopecia: Pathogenesis and Potential for Therapy, Expert Rev. Mol. Med. (2002) 4(22): 1-11; Norwood O. T., Hair Transplant Surgery, Springfield, Ill. (Charles C. Thomas Publishers) 1973:3-16; Rajput R. J., Controversy: Is There a Role for Adjuvants in the Management of Male Pattern Hair Loss? Journal of Cutaneous and Aesthetic Surgery. (2010) 3(2): 82-86. Females can also experience androgenic alopecia to some degree, although its manifestation in terms of the loci of hair loss is different than that observed in men. Olsen E. A., Hordinsky M., Whiting D., Stough D., Hobbs S., Ellis M., Wilson T., Rittmaster R. S., The Import ice of Dual 5 Alpha Reductase Inhibition in the Treatment of Male Pattern Hair Loss: Results of a Randomized Placebo-Controlled Study of Dutasteride Versus Finasteride, Journal of the American Academy of Dermatology (2006) 55(6): 1014-1023; Olsen E., Pattern Hair Loss. In: Disorders of hair Growth. Diagnosis and Treatment, New York: McGraw Hill (2003) 321-362; Sinclair R., Male Pattern Androgenetic Alopecia, British Medical J. (1998) 317: 865-869; Whiting D. A., Male Pattern Hair Loss: Current Understanding, Int. J. Dermatol. (1998) (37): 561-566; Wilson J. D., Griffin J. E., Russell D. W., Steroid 5 Alpha Reductase 2 Deficiency, Endocr. Rev. (1993) (14): 577-593.

Male pattern baldness is thought to be caused by a genetic sensitivity of hair follicles to dihydrotestosterone, which causes the hair follicles to shrink. This is believed to shorten their lifespan and their anagen (growth) phase and prevent them from producing hair normally.

More specifically, hair growth begins under the skin in structures called follicles. Each strand of hair normally grows for approximately 2 to 6 years, goes into a resting phase for several months, and then falls out. The cycle starts over when the follicle begins growing a new hair. Increased levels of androgens, particularly dihydrotestosterone, in hair follicles can lead to a shorter cycle of hair growth and the growth of shorter and thinner strands of hair. Additionally, there is a delay in the growth of new hair to replace strands that are shed.

Men with male pattern baldness typically have higher levels of 5-alpha reductase, lower levels of total testosterone, higher levels of unbound/free testosterone, and higher levels of total free androgens including dihydrotestosterone. As noted above, 5-alpha reductase is known to have to isoforms—Type I and Type II. Notably, men who have a congenital deficiency of the Type II isozyme do not experience male pattern baldness.

Males who have male pattern baldness typically observe hair loss in a well-defined pattern, beginning above both temples. Over time, the hairline recedes to form a characteristic “M” shape. Hair also thins at the crown (near the top of the head), often progressing to partial or complete baldness. The pattern of hair loss in women differs from male pattern baldness. In women, the hair becomes thinner all over the head, but the hairline does not recede. Androgenic alopecia in women rarely leads to total baldness.

At present, most treatment modalities for male pattern baldness are not FDA approved and overall are not particularly effective. The high prevalence of male pattern baldness, the typically early age of onset, and the large degree of associated psychosocial symptoms have created a large market for treatment of male pattern baldness. Despite the paramount demand for male pattern baldness treatment, there are only two FDA approved medications. However, they are costly and may have side effects.

The two FDA approved medications used to treat male pattern baldness include topical minoxidil (Rogaine®; Johnson & Johnson) and finasteride (Propecia®; Merck). Minoxidil is a vasodilator which is directly applied to the scalp to stimulate growth of the hair follicles. Topical minoxidil slows hair loss for many men, while in some men it grows new hair. The previous degree of hair loss returns when solution application is discontinued. Minoxidil is available in 2% and 5% solutions, and a 5% foam version that became available in 2007.

Propecia® 1 mg (oral) was approved by the FDA in 1998 for androgenic alopecia. Propecia® (finasteride), is a prescription pill that inhibits the production of dihydrotestosterone (DHT), which is accomplished primarily by blocking the activity of the Type II isoform of 5-alpha reductase. Like minoxidil, finasteride is likely to slow hair loss, and may also stimulate new hair growth. In general, finasteride is somewhat more effective than minoxidil, and it is more effective when combined with minoxidil. As with minoxidil, one's previous degree of hair loss returns when finasteride is discontinued.

There are other medications available on the market that claim to treat male pattern baldness. These medications, however, are not FDA approved for treating male pattern baldness. Ketoconazole (Nizoral®; Johnson & Johnson) shampoo, is known to inhibit 5-alpha reductase, Preliminary research suggests that ketoconazole shampoo may be beneficial in men suffering from androgenic alopecia. Downs M., Nizoral Shampoo/or Hair Loss, DERMAdoctor, www.dermadoctor.com (2011); Piérard-Franchimont C., De Doncker P., Cauwenbergh G., Piérard G. E., Ketoconazole Shampoo: Effect of Long-Term Use in Androgenic Alopecia, Dermatology (1998) 196(4): 474-477. Support for this also sterns from a study in 1998 which compared ketoconazole shampoo 2% to the proven hair loss drug minoxidil in men with androgenic alopecia. Piérard-Franchimont C., De Doncker P., Cauwenbergh G., Piérard G. E., Ketoconazole Shampoo: Effect of Long-Term Use in Androgenic Alopecia, Dermatology (1998) 196(4): 474-477. The latter study concluded that hair density, size, and proportion of anagen follicles were improved almost similarly by both ketoconazole and 2% minoxidil regimens. In addition, since ketoconazole effectively treats the Pityriasis (also called Malassezia) fungus that commonly inhabits the scalp, it was hypothesized that it may prevent hair loss by reducing inflammation from the fungus, in addition to having anti-androgenic properties. The researchers were guarded about the meaning of their results. Although applicants are unaware of any further research in humans has been undertaken, a study on ketoconazole in 2005 corroborated the existence of a stimulatory effect on hair growth in mice, Jiang J., Tsuboi R., Kojinia Y., Ogawa H., Topical application of ketoconzazole stimulates hair growth in C.3H/HeN mice, J. Dermatol. 32(4): 243-247 (April 2005).

In patients who are atopic (allergic hypersensitivities), there is an increased susceptibility to infection or colonization with various organisms. For example, Staphylococcus aureus is detected in more than 90% of skin lesions of atopic dermatitis. A superimposed dermatophytosis including Trichophyton rubrum and Malassezia furfur (Pityrosporum ovate) can cause flares of atopic dermatitis. The rate of isolation of Malassezia from the skin of atopic dermatitis patients is much higher than from the skin of healthy control patients without atopy. This fungal (yeast) organism is also commonly associated with concomitant seborrheic dermatitis in atopic patients. Malassezia furfur is a lipophilic yeast, and anti-yeast IgE antibodies have been found in patients with a predominant head and neck eczematous dermatitis. This common presentation in atopic dermatitis patients provided the interest in studying patients with clinical fungal infection of the head and neck. And, it suggests a correlation of a superimposed fungal infection of the scalp in patients with seborrheic dermatitis and male pattern baldness. Hiruma M., Maeng D. J., Kobayashi M., Suto H., Ogawa H., Fungi and Atopic Dermatitis, Nippon Ishinkin Gakkai Zasshi (1999) 40(2): 79-83.

Another important medication which inhibits 5-alpha reductase is dutasteride, (Avodart®; GSK). There has been significant attention towards dutasteride as it is believed to be the only well-known medication which blocks both the Type I and Type II isozymes of 5-alpha reductase. Type I 5-alpha reductase is the principle isozyme found in sebaceous and sweat glands and the scalp. Olsen E. A., Hordinsky M., Whiting D., Stough D., Hobbs S., Ellis M., Wilson T., Rittmaster R. S., The Importance of Dual 5 Alpha Reductase Inhibition in the Treatment of Male Pattern Hair Loss: Results of a Randomized Placebo-Controlled Study of Dutasteride Versus Finasteride, Journal of the American Academy of Dermatology (2006) 55(6): 1014-1023; Chul Eun H., Sang Kwon O., Ho Yeon J., Seung Shin H., Yoon Kim B., In Ro B., Kyon H., Young Sim W., Lynn Lew B., Lee W. S., Young Park H., Phil Hong S., Hong Ji J., Efficacy, Safety, and Tolerability of Dutasterie 0.5 mg Once Daily in Male Patients with Male Pattern Hair Loss: A Randomized, Double Blind, Placebo-Controlled, Phase III Study, Journal of the American Academy of Dermatology (2010) 63(2): 252-258; Sato T., Sonoda T., Itami S., Takayasu S., Predominance of Type I 5 Alpha Reductase in Apocrine Sweat Glands of Patients With Excessive or Abnormal Odour Derived From Apocrine Sweat (osmidrosis), Br. J. Dermatol. (1998) 139: 806-810; Thiboutot D., Harris G., Iles V., Cimis G., Gilliland K., Hagari S., Activity of the Type I 5 Alpha-Reductase Exhibits Regional Differences in Isolated Sebaceous Glands and Whole Skin, J. Invest. Dermatol. (1995) 105: 209-214; Jenkins G. P., Andersson S., Imperato-McGinley J., Wilson J. D., Russell D. W., Genetic and Pharmacologic Evidence for More Than One Human Steroid 5 Alpha-Reductase, J. Clin. Invest. (1992) 89: 293-300; Russell D. W., Wilson J D., Steroid 5 Alpha-Reductase: Two Genes/Two Enzymes, Ann. Rev. Biochem. (1994) 63: 25-61; Thigpen A. E., Silver R. I., Guileyard J. M., Casey M. L., McConnell J. D., Russell D. W., Tissue Distribution and Ontogeny of Steroid 5 Alpha-Reductase Isozyme Expression, Invest. (1993) 92: 903-910. Whereas, Type II 5-alpha reductase is present in hair follicles and the prostate. Chul Eui H., Sang Kwon O., Ho Yeon J., Seung Shin H., Yoon Kim B., In Ro B., Kyon H., Young Sim W., Lynn Lew B., Lee W. S., Young Park H., Phil Hong S., Hong Ji J., Efficacy, Safety, and Tolerability of Dutasteride 0.5 mg Once Daily in Male Patients Wale Pattern Hair Loss: A Randomized, Double Blind, Placebo-Controlled, Phase III Study, Journal of the American Academy of Dermatology (2010) 63(2): 252-258.

In addition to blocking both isozymes of 5-alpha reductase, dutasteride has been shown to be 3 times more potent than finasteride at inhibiting the Type II isozyme of 5-alpha reductase, and more than 100 times as potent at inhibiting the type I isozyme. Olsen E. A., Hordinsky M., Whiting D., Stough D., Hobbs S., Ellis M., Wilson T., Rittmaster R. S., The Importance of Dual 5 Alpha Reductase Inhibition in the Treatment of Male Pattern Hair Loss: Results of a Randomized Placebo-Controlled Study of Dutasteride Versus Finasteride, Journal of the American Academy of Dermatology (2006) 55(6): 1014-1023, Oral dutasteride also decreases serum dihydrotestosterone by up to 90%, whereas finasteride reduces concentrations of serum dihydrotestosterone by about 70%. Olsen E. A., Hordinsky M., Whiting D., Stough D., Hobbs S., Ellis M., Wilson T., Rittmaster R. S., The Importance of Dual 5 Alpha Reductase Inhibition in the Treatment of Male Pattern flair Loss: Results of a Randomized Placebo-Controlled Study of Dutasteride Versus Finasteride, Journal of the American Academy of Dermatology (2006) 55(6): 1014-1023; Clark R. V., Hermann D. J., Cunningham G. R., Wilson T. H., Morrill B. B., Hobbs S., Marked Suppression of Dihydrotestosterone in Men with Prostatic Hyperplasia by Dutasteride, a Dual 5 Alpha-Reductase Inhibitor, J. Endocrinol. Metab. (2004) 89: 2179-2184. These properties potentially make dutasteride a desirable candidate for treating male pattern baldness. Despite these more desirable properties, limited research using dutasteride for male pattern baldness treatment has been undertaken.

Initially, phase I and II clinical trials for dutasteride as a hair loss drug were undertaken, but called off in late 2002. The reason these trials were called off is not publicly known. Industry sources speculate that dutasteride would have been seen as too similar to Propecia® (1 mg/day finasteride) to have been proven profitable on the market as a hair loss treatment. Olsen E. A., Hordinsky M., Whiting D., Stough D., Hobbs S., Ellis M., Wilson T., Rittmaster R. S., The importance of Dual 5 Alpha Reductase Inhibition in the Treatment of Male Pattern Hair Loss: Results of a Randomized Placebo-Controlled Study of Dutasteride Versus Finasteride, Journal of the American Academy of Dermatology (2006) 55(6): 1014-1023. Phase II study results, however, indicated that oral dutasteride at both 0.5 mg and 2.5 mg/day generated a superior hair count to 5 mg/day finasteride at 12 and 24 weeks. Olsen E. A., Hordinsky M., Whiting D., Stough D., Hobbs S., Ellis M., Wilson T., Rittmaster R. S., The Importance of Dual 5 Alpha Reductase Inhibition in the Treatment of Male Pattern Hair Loss: Results of a Randomized Placebo-Controlled Study of Dutasteride Versus Finasteride, Journal of the American Academy of Dermatology (2006) 55(6): 1014-1023.

The foregoing demonstrates that while treatments for male pattern baldness exist, their efficacy is limited. Thus, an improved treatment regimen is needed.

### BRIEF SUMMARY OF THE INVENTION

Given the lack of a strongly and rapidly efficacious treatment for male pattern baldness, an object of the invention set forth herein was to develop a highly efficacious treatment for make pattern baldness that produces rapid and striking results.

A further object of the invention set forth herein was to develop a highly efficacious treatment for make pattern baldness that produces rapid and striking results where said treatment takes into account the additional features presented by atopic subjects having male pattern baldness.

Accordingly, a treatment regimen has been developed, as described herein, in which highly efficacious and rapid results are achieved in both atopic and non-atopic subjects.

One aspect of one such treatment regimen of the invention described herein comprises a composition that includes pharmaceutically effective amounts of minoxidil, finasteride, and dutasteride formulated into a cream to be topically applied to the scalp of subjects suffering from androgenic alopecia. “Cream,” as used herein, may constitute a cream, lotion, ointment, or other compositions of similar consistency.

Another aspect of a treatment regimen of the invention described herein comprises oral administration of pharmaceutically effective amounts of finasteride in conjunction with topical application to the scalp of the above-referenced cream-based composition.

Yet another aspect of a treatment regimen of the invention described herein comprises topical application to the scalp of pharmaceutically effective amounts of minoxidil foam in conjunction with the topical application to the scalp of the above-referenced cream-based composition.

A further aspect of a treatment regimen of the invention described herein comprises use of pharmaceutically effective amounts of ketoconazole shampoo in conjunction with the topical application to the scalp of the above-referenced cream-based composition.

Yet a further aspect of one treatment regimen of the invention described herein comprises topical application to the scalp of the above-referenced cream-based composition in conjunction with oral administration of pharmaceutically effective amounts of finasteride and topical application to the scalp of pharmaceutically effective amounts of minoxidil foam.

Yet a further aspect of another such treatment regimen of the invention described herein comprises topical application of the above-referenced cream-based composition in conjunction with oral administration of pharmaceutically effective amounts of finasteride and use of pharmaceutically effective amounts of ketoconazole shampoo.

Yet a further aspect of a treatment regimen of the invention described herein comprises topical application to the scalp of the above-referenced cream-based composition in conjunction with topical application to the scalp of pharmaceutically effective amounts of minoxidil foam and use of pharmaceutically effective amounts of ketoconazole shampoo.

Yet a further aspect of a treatment regimen of the invention described herein comprises topical application to the scalp of the above-referenced cream-based composition in conjunction with oral administration of pharmaceutically effective amounts of finasteride, topical application to the scalp of pharmaceutically effective amounts of minoxidil foam, and use of pharmaceutically effective amounts of ketoconazole shampoo.

Yet a further aspect of the treatment regimen of the invention described herein comprises use of hypoallergenic ingredients for the cream base of the above-referenced composition so as to minimize any potential affects of allergic inflammation on hair growth in atopic subjects.

In one embodiment of the treatment regimen of the invention described herein, the above-referenced cream-based composition is preferably applied once per day.

In another embodiment of the treatment regimen of the invention described herein, finasteride is orally administered preferably once per day.

In another embodiment of the treatment regimen of the invention described herein, the minoxidil foam is applied preferably at least once per day.

In another embodiment of the treatment regimen of the invention described herein, the ketoconazole shampoo is preferably used 2-3 times per week, preferably lathered onto the scalp for 90-120 seconds, and preferably allowed to remain on the scalp for 10 minutes before washing it out.

In the subjects studied, with assessment of hair growth based on the Hamilton Norwood Scale, all subjects demonstrated significant hair regrowth. Those using only the above-referenced cream-based composition demonstrated significant hair regrowth within an average of 90 days, although several subjects demonstrated significant hair regrowth in as little as 30-60 days. Those using all four components of the treatment regimen (cream-based composition, oral finasteride, minoxidil foam, and ketoconazole shampoo) demonstrated significant hair regrowth in an average of 30 days, which is believed to be much faster than any current treatment regimen on the market. One subject showed significant hair regrowth in as little as 14 days. Inasmuch as none of the ingredients in the treatment regimen of the invention described herein alone are believed to significantly stimulate hair growth in 14-30 days, the combined effect of the components in the treatment regimen of the invention described herein, and their manner of administration, is a synergistic one in which the results are not merely the sum of each component, and in which the outcome was not predictable based on the properties of each individual component of the composition. Furthermore, there was nothing about the combination of active ingredients that suggested that an accelerated and more robust effect would be observed with the combination relative to each active ingredient alone.

In prior art treatment regimens of 5% rogaine with oral Propecia®, hair regrowth, if it appears at all, typically requires 4-6 months to occur. The primary effect of a 5% Rogaine®/oral Propecia® regimen is a slowing of hair loss. The present invention not only slowed hair loss, but provided for rapid and significant hair growth, which appears to be a vast improvement over prior art treatment methods.

Particularly noteworthy is the effect produced in subjects with atopic dermatitis. In patients utilizing all four components of the treatment regimen, not only was the dermatitis resolved, but the hair regrowth was significant and rapid (average: 30 days). Even if the minoxidil foam was not used, the dermatitis resolved and significant hair regrowth occurred in an average of 30 days. In one subject who only used the above-referenced cream-based composition and the ketoconazole shampoo, the dermatitis resolved and significant hair regrowth occurred in 60 days. Thus, the present treatment regimen may be particularly effective in subjects with male pattern baldness and atopic dermatitis on their scalps.

Accordingly, the composition and methods for treating hair loss described herein, including topical application of the cream-based composition, produced a synergistic, highly efficacious, and rapid effect that could not have been predicted by merely knowing and understanding the individual components of the treatment regimen.

### DETAILED DESCRIPTION OF THE INVENTION

The detailed description set forth below, or elsewhere herein, including any charts, tables, or figures, is intended as a description of presently-preferred embodiments of the invention and is not intended to represent the only forms in which the present invention may be constructed or utilized, nor is it intended to limit the scope of any claims based thereon.

The invention described herein is directed to novel methods and a novel composition for treatment of hair loss by inducing hair growth in individuals suffering from androgenic alopecia (a/k/a male pattern baldness). Specifically, the composition described herein is a component of a larger regimen for treating hair loss.

The composition preferably comprises 2.0 mg of dutasteride (Avodart®), 10 mg of Finasteride (Proscar®), 20 ml of sterile water, 21 ml of Vanicream® hypoallergenic moisturizing skin cream, and 8 ml of 5% minoxidil (Rogaine®). This combination of products comes to a total of 50 ml, but can easily be scaled to larger or lesser amounts.

The composition is made as follows: approximately 2.0 mg of Avodart®, (dutasteride) and approximately 10 mg of Proscar® or Propecia® (finasteride) were mixed at room temperature for approximately 24-48 hours in approximately 20 ml of sterile water using a standard laboratory warier. After approximately 24-48 hours, approximately 8 ml of 5% Rogaine® (minoxidil) solution and approximately 21 ml of Vanicream® hypoallergenic moisturizer were added to the solution and came to a total of approximately 50 ml. This combination was mixed at room temperature on the swirler for an additional approximately 24-72 hours. The combination was blended into a cream or light lotion for subsequent application to the scalp of a subject. The finished composition was stored under refrigeration. It was noted that the generic forms of dutasteride and finasteride did not perform as well as the branded forms, Where drug names are used herein, a generic name is deemed to include both generic and branded forms, and a brand name is deemed to include both branded and generic forms, unless the context clearly indicates otherwise.

Expressed by weight concentration or percentage, the composition comprises approximately 0.04 mg/ml Avodart® (dutasteride), 0.2 mg/ml Proscar® (finasteride), 40% water, 42% Vanicream® hypoallergenic moisturizer, and 0.8% Rogaine® (minoxidil) (active ingredient only included in the percentage).

Vanicream® hypoallergenic moisturizer is a proprietary blend of the following ingredients: purified water, white petrolatum, sorbitol solution, cetearyl alcohol, propylene ceteareth-20, simethicone, glyceryl monostearate, polyethylene glycol monostearate, sorbic acid, and butylatedhydroxytoluene (BHT).

Other suitable hypoallergenic creams and moisturizers available on the market can be substituted for the Vanicream® hypoallergenic moisturizer. A representative example of such a substitute is SPF-15 Facial Moisturizer from 220 Laboratories in Riverside, Calif. The contents of SPF-15 Facial Moisturizer (Product Code 162-98A) from 220 Laboratories consists of the following ingredients in a proprietary blend: zinc; oxide, water, caprylic/capric triglyceride, glycerin, cetearyl glucoside, cetearyl alcohol, stearyl alcohol, xanthan gum, phenoxyethanol, benzyl alcohol, glyceryl isostearate, polyhydroxystearic acid, tocopherol acetate, chamonilla recutica (matricaria) flower extract, alow barbadensis (aloe vera) leaf extract, butyrospermum parkii (shea butter), with or without fragrance.

Applicants do not intend to be limited to the foregoing examples of hypoallergenic creams and moisturizers as a vehicle for their novel composition, and incorporate by reference other compositions that comprise creams, lotions, and/or ointments known in the art, that facilitate compounding, that are widely compatible with active ingredients, and that are hypoallergenic.

Pharmaceutically effective amounts of other suitable hair loss treatment agents, such as derivatives of finasteride and dutasteride, or compounds with similar properties and/or functions (e.g., inhibition of Type I 5-alpha reductase, Type II 5-alpha reductase, or both), as described, for example, in U.S. Pat. No. 4,139,619 to Chidsey III, U.S. Pat. No. 4,760,071 to Rasmusson, U.S. Pat. No. 5,525,608 to Adams, U.S. Pat. No. 5,516,779 to Von Langen, and U.S. Pat. No. 5,763,361 to Harris, could conceivably be used in place of finasteride and/or dutasteride in the composition described herein.

The following examples are representative examples from four different treatment protocol groups.

### Example 1

A subject suffering from androgenic alopecia or male pattern baldness applied the composition to his scalp once per day; orally ingests Propecia® (1 mg) once per day; applies Rogaine® (minoxidil) 5% foam to his scalp at least once per day; and, shampoos his (or her) hair and scalp with a shampoo containing 2% ketoconazole 2-3 times per week where the subject lathers or scrubs the ketoconazole-containing shampoo into his (or her) scalp for approximately 90-120 seconds and allows the foam to remain on the scalp for approximately 10 minutes prior to rinsing. The results of this treatment regimen are referenced in the Tables below and further shown in the attachment to the related provisional application (U.S. Provisional Patent Application Ser. No. 61/472,567 filed Apr. 6, 2011), which is incorporated herein by reference.

### Example 2

A subject suffering from androgenic alopecia or male pattern baldness applies the composition to his (or her) scalp once per day; orally ingests Propecia® (1 mg) once per day; and, shampoos his (or her) hair and scalp with a shampoo containing 2% ketoconazole 2-3 times per week where the subject lathers or scrubs the ketoconazole-containing shampoo into his (or her) scalp for approximately 90-120 seconds and allows the foam to remain on the scalp for approximately 10 minutes prior to rinsing. The results of this treatment regimen are referenced in the Tables below and further shown in the attachment to the related provisional application (U.S. Provisional Patent Application Ser. No. 61/472,567 filed Apr. 6, 2011)which is incorporated herein by reference.

### Example 3

A subject suffering from androgenic alopecia or male pattern baldness applies the composition to his (or her) scalp once per day, and shampoos his (or her) hair and scalp with a shampoo containing 2% ketoconazole 2-3 times per week where the subject lathers or scrubs the ketoconazole-containing shampoo into his (or her) scalp for approximately 90-120 seconds and allows the foam to remain on the scalp for approximately 10 minutes prior to rinsing. The results of this treatment regimen are referenced in the Tables below and further shown in the attachment to the related provisional application (U.S. Provisional Patent Application Ser. No. 61/472,567 filed Apr. 6, 2011), which is incorporated herein by reference.

### Example 4

A subject suffering from androgenic alopecia or male pattern baldness applies the composition to his (or her) scalp once per day. The results of this treatment regimen are referenced in the Tables below and further shown in the attachment to the related provisional application (U.S. Provisional Patent Application Ser. No. 61/472,567 filed Apr. 6, 2011), which is incorporated herein by reference.

Additional data related to the foregoing examples, and which includes the foregoing examples, is shown in Tables 1 and 2 below. Examples 1-4 above correspond to Groups 1-4 in the Tables below.

TABLE 1
Subject Groups

| | Group 1 | Group 2 | Group 3 | Group 4 |
|---|---|---|---|---|
| Components | Novel | Novel | Novel | Novel |
| | composition | composition | composition | composition |
| | described | described | described | described |
| | herein; | herein; | herein; | herein |
| | finasteride | finasteride | ketoconazole | |
| | (1 mg/day); | (1 mg/day); | shampoo 2-3 | |
| | minoxidil foam | ketoconazole | times/week | |
| | (once/day); | shampoo 2-3 | | |
| | ketoconazole | times/week | | |
| | shampoo 2-3 | | | |
| | times/week | | | |
| # of Subjects | 8 | 1 | 1 | 5 |
| Presence of | 8 | 1 | 1 | 0 |
| Seborrheic | | | | |
| Dermatitis | | | | |
| Average | 30 days | 30 days | 60 days | 90 days |
| Time for | | | | |
| Hair | | | | |
| Regrowth | | | | |
| (Days) | | | | |

TABLE 2
Subject Demographics and Response to Treatment

| | | | | Hamilton |
|---|---|---|---|---|
| | | | Atopic | Norwood |
| Subject | Group | Age | (Y/N) | Scale |
| 1 | 4 | 33 | N | IV |
| 2 | 4 | 38 | N | IV/V |
| 3 | 1 | 24 | Y | VII |
| 4 | 1 | 38 | Y | II |
| 5 | 4 | 50 | N | III |
| 6 | 3 | 38 | Y | V |
| 7 | 1 | 43 | Y | VII |
| 8 | 4 | 72 | N | III |
| 9 | 1 | 42 | Y | VII |
| 10 | 1 | 32 | Y | VII |
| 11 | 2 | 38 | N | III |
| 12 | 4 | 45 | Y | VII |
| 13 | 1 | 30 | Y | V |
| 14 | 1 | 59 | Y | II |
| 15 | 1 | 36 | Y | IV |

With respect to each of the foregoing examples, where the subjects used the ketoconazole-containing shampoo, instances of dermatitis were significantly improved typically within approximately thirty (30) days of commencement of treatment under the foregoing methods.

With respect to each of the foregoing examples, the steps are to be performed, as indicated, indefinitely (i.e., without any cessation), While further study of the foregoing methods may demonstrate that treatment may be ceased at some point without losing the beneficial effects, the issue of duration of treatment has yet to be resolved. Thus, the methods employed herein are to be practiced by a subject for as long as he (or she) desires the results of the treatment.

With respect to each of the foregoing examples, pharmaceutically effective amounts of anti-fungal agents (other than ketoconazole) (e.g., miconazole) that can be applied as a shampoo or in a cream, lotion, or ointment, and are believed to prevent hair loss and/or promote hair re-growth, may be substituted for ketoconazole to the extent applicable.

With respect to the foregoing examples, one may envision use of the composition wherein Avodart® (dutasteride) is omitted from the composition without a meaningful change in the results provided that the remaining steps of the foregoing examples are practiced, particularly, but not exclusively, with respect to those steps in Example 1 above. The same may be true with respect to Propecia® (finasteride).

With respect to the foregoing examples, various combinations of steps from the foregoing examples may be envisioned as potential treatments for androgenic alopecia or male pattern baldness while keeping with the spirit of the invention described herein. For example, a subject may use the composition more than once per week but less than daily. In addition, a subject may use various combinations of the steps involving Propecia®, Rogaine®, and/or the ketoconazole-containing shampoo in conjunction with the composition. Furthermore, a subject may vary the frequency of the use of the steps involving Propecia®, Rogaine®, and the ketoconazole-containing shampoo; provided, that the use is frequent enough and the dosage effective to produce rapid hair re-growth when used in conjunction with the composition, as described herein. Such hair growth was substantially more rapid than use of 5% Rogaine® and 1 mg/day Propecia®, which typically slows hair loss rather than significantly stimulating hair re-growth. The results of the methods described herein for treatment of hair loss with the composition with or without the other components described herein provide for more rapid, more efficacious, and more potent results that are not merely the sum of their parts, but are synergistic, unexpected, and robust.

The foregoing descriptions of the preferred embodiments of the invention have been presented for the purposes of illustration and description only. They are not intended to be exhaustive or to limit the invention to the precise form(s) disclosed. Many modifications and variations are possible in light of the above teaching and in keeping with the spirit of the invention described herein. It is intended that the scope of the invention not be limited by this specification, but only by the claims and the equivalents to the claims appended hereto.

What is claimed is:

1. A composition for treating hair loss and stimulating hair growth comprising:
a. a pharmaceutically effective amount of a first inhibitor of Type I 5-alpha reductase, Type II 5-alpha reductase, or both;
b. a pharmaceutically effective amount of a second inhibitor of Type I 5-alpha reductase, Type II 5-alpha reductase, or both;
c. a pharmaceutically effective amount of a vasodilator that reduces hair loss and/or stimulates hair growth;
d. approximately 30-60% by volume of a cream vehicle; and
e. approximately 30-60% by volume water.

2. The composition for treating hair loss and stimulating hair growth of claim 1, wherein the first inhibitor of Type I 5-alpha reductase, Type II 5-alpha reductase, or both comprises finasteride.

3. The composition for treating hair loss and stimulating hair growth of claim 1, wherein the second inhibitor of Type I 5-alpha reductase, Type II 5-alpha reductase, or both comprises dutasteride.

4. The composition for treating hair loss and stimulating hair growth of claim 1, wherein the vasodilator comprises minoxidil.

5. The composition for treating hair loss and stimulating hair growth of claim 1, wherein the cream vehicle is hypoallergenic.

6. The composition for treating hair loss and stimulating hair growth of claim 2, wherein the second inhibitor of Type I 5-alpha reductase, Type II 5-alpha reductase, or both comprises dutasteride.

7. The composition for treating hair loss and stimulating hair growth of claim 2, wherein the vasodilator comprises minoxidil.

8. The composition for treating hair loss and stimulating hair growth of claim 3, wherein the vasodilator comprises minoxidil.

9. The composition for treating hair loss and stimulating hair growth of claim 6, wherein the vasodilator comprises minoxidil.

10. The composition for treating hair loss and stimulating hair growth of claim 2, wherein the cream vehicle is hypoallergenic.

11. The composition for treating hair loss and stimulating hair growth of claim 3, wherein the cream vehicle is hypoallergenic.

12. The composition for treating hair loss and stimulating hair growth of claim 4, wherein the cream vehicle is hypoallergenic.

13. The composition for treating hair loss and stimulating hair growth of claim 6, wherein the cream vehicle is hypoallergenic.

14. The composition for treating hair loss and stimulating hair growth of claim 9, wherein the cream vehicle is hypoallergenic.

15. A method for making the composition of claim 14, said method capable of upward or downward scaling, comprising:
a. mixing approximately 0.1 mg/ml dutasteride and approximately 0.5 mg/ml finasteride in approximately 20 ml of water at room temperature for approximately 24-48 hours;
b. adding approximately 8.0 ml of 5% minoxidil solution and approximately 21 ml of hypoallergenic cream vehicle to the 0.1 mg/mi dutasteride and approximately 0.5 mg/mi finasteride in approximately 20 ml of water for a combination totaling approximately 50 ml;
c. mixing the combination for approximately 24-72 hours;
d. blending the combination into a cream; and,
e. storing the composition under refrigeration for subsequent use.

16. A method for treating hair loss and stimulating hair growth comprising:
applying to a scalp of a subject at least once every other day the composition of claim 1.

17. The method for treating hair loss and stimulating hair growth of claim 16, further comprising:
applying to the scalp of the subject, at least twice-weekly, an anti-fungal shampoo.

18. The method for treating hair loss and stimulating hair growth of claim 16, further comprising:
ingesting orally, once daily, a 5-alpha reductase inhibitor.

19. The method for treating hair loss and stimulating hair growth of claim 16, further comprising:
applying to the scalp of the subject, at least thrice-weekly, minoxidil foam.

20. The method for treating hair loss and stimulating hair growth of claim 17, wherein the anti-fungal shampoo is ketoconazole.

21. The method for treating hair loss and stimulating hair growth of claim 18, wherein the 5-alpha reductase inhibitor is finasteride.

22. The method for treating hair loss and stimulating hair growth of claim 19, wherein the anti-fungal shampoo is ketoconazole.

23. The method for treating hair loss and stimulating hair growth of claim 19, wherein the 5-alpha reductase inhibitor is finasteride.

24. The method for treating hair loss and stimulating hair growth of claim 20, wherein the 5-alpha reductase inhibitor is finasteride.

25. The method for treating hair loss and stimulating hair growth of claim 22, wherein the 5-alpha reductase inhibitor is finasteride.
